(19) United States
(12) Patent Application Publication
(10) Pub. No.: US 2004/0024044 A1
(43) Pub. Date: Feb. 5, 2004

(54) Exemestane as chemopreventing agent

(76) Inventors: Enrico Di Salle, Milan (IT); Gabriella Piscitelli, Milan (IT); Giorgio Massimini, Abbiategrasso Milan (IT); Dinesh Purandare, Branchburg, NJ (US); Alessandro Martini, Milan (IT); Lorena Muggetti, Meda (IT)

Correspondence Address: Stephen H Docter, McDonnell Boehnen Hulbert & Berghoff, Suite 3200, 300 South Wacker, Chicago, IL, 60606, US

(21) Appl. No.: 10/363,935

(22) PCT Filed: Aug. 31, 2001
(86) PCT No.: PCT/EP01/10172

(51) Int. Cl. A61K 31/404

(52) U.S. Cl. 514/414

(57) ABSTRACT

The present invention concerns the use of aromatase inhibitor exemestane, either alone or in combination with other therapeutic agents in the chemoprevention of estrogen dependent cancer in mammals, including humans, at increased risk of the disease.

### FIELD OF THE INVENTION

The invention belongs to the fields of pharmaceutical chemistry and anti-cancer medicine, and provides a method of chemoprevention of estrogen dependent cancer.

### BACKGROUND OF THE INVENTION

Cancers, including estrogen dependent cancers, are generally thought to result from a multistep process, in which a series of somatic mutations, and/or chromosomal changes occur. Each step results in a greater deviation from normal cellular behavior, until cells lose the normal ability to regulate their own growth and therefore proliferate. The altered cells first proliferate into a precanceruos neoplasm, which progresses in stages toward metastatic cancer. This process is known as tumor progression. On the other hand, for instance approximately 30% of breast cancers are hormone-sensitive and are treated with a variety of agents other than oophorectomy (surgical or radiological), including anti-estrogens, progestins and aromatase inhibitors. Despite the variety of treatments available, approximately on third of the early treated breast cancer (EBC) will relapse within 10 years from diagnosis, and as soon as the disease becomes metastatic (BMC), the medium life expectancy is of about 2,5-3 years. There is therefore a high and unmet medical need for therapeutic agents aimed at prevention of hormone dependent tumors and, in particular, of both primary and secondary breast cancer.

Cancer chemoprevention is a new discipline whose foundation rests upon epidemiologic evidence suggesting that dietary components including vitamins and micronutrients such as beta-carotene, vitamin E, calcium and selenium may be inhibitors of carcinogenesis. However, although the precise biological mechanisms of cellular carcinogenesis are incomplete, a number of specific mechanisms seem to be procarcinogenic. Accordingly, estrogen modulators for instance may act as a chemopreventive agents in breast cancer by disrupting estrogen production, receptor binding or receptor activation. In this connection, the chemopreventive properties of tamoxifen were first demonstrated by the reduction of second primaries in a meta-analysis of breast cancer survivors who had taken the drug for 5 years. A major concern remains, however: the increased risk of endometrial cancer associated with tamoxifen administration. Since chemopreventive agents are intended for chronic (or long lasting) use in healthy or relative healthy subjects, toxicity, even if mild and reversible, is problematic. Accordingly, there is the need in this field of drugs endowed with low side effects and combinations of anticancer agents with non-overlapping toxicity while having enhanced therapeutic effect.

### SUMMARY OF THE INVENTION

The present invention concerns the use of aromatase inhibitor exemestane in the chemoprevention of estrogen dependent cancer in mammals, including humans, at increased risk of the disease, either alone or in combination with additional therapeutic agents.

### DETAILED DESCRIPTION

The present invention provides as a first object the use of exemestane in the manufacture of a medicament for chemoprevention or controlling the growth of estrogen dependent cancer.

The present invention also provides the use of exemestane in the manufacture of a medicament for chemoprevention or controlling the growth of estrogen dependent cancer, in a patient undergoing a simulataneous, separate or sequential treatment with another chemopreventive agent selected from a taxane compound, a non-steroidal anti-inflammatory compound (NSAID), a retinoid compound, a farnesyl-protein transferase inhibitor, a matrix metalloprotease inhibitor, an αvβ3 integrin inhibitor, an anthracycline compound, an antibody against HER2, and EGFR antagonist or inhibitor, a protein kinase inhibitor, linomide, angiostatin, dehydroepiandrosterone (DHEA), a telomerase inhibitor, a cyclooxygenase inhibitor, razoxin, platelet factor 4 (endostatin), a VEGF inhibitor, an anti-estrogen and thalidomide, or a mixture thereof.

A further object of the invention is to provide a method for chemopreventing or controlling the growth of estrogen dependent cancer in a mammal in need of such treatment, including humans, comprising administering to said mammal a therapeutically effective amount of exemestane.

The invention in addition provides a combined method of chemoprevention or of controlling the growth of estrogen dependent cancer in a mammal in need of such treatment, including humans, comprising administering simultaneous, separately or sequentially to said mammal, exemestane, and another chemopreventive agent selected from a taxane compound, a non-steroidal anti-inflammatory compound (NSAD), a retinoid compound, a farnesyl-protein transferase inhibitor, a matrix metalloprotease inhibitor, an αvβ3 integrin inhibitor, an anthracycline compound, an antibody against HER2, and EGFR antagonist or inhibitor, a protein kinase inhibitor, linomide, a cyclooxygenase inhibitor, razoxin, angiostatin, dehydroepiandrosterone (DHEA), a telomerase inhibitor, platelet factor 4 (endostatin), an anti-estrogen, a VEGF inhibitor and thalidomide, or a mixture thereof; in amounts and close in time sufficient to produce a therapeutically useful effect.

The invention also provides a product containing exemestane and another chemopreventive agent selected from a taxane compound, a non-steroidal anti-inflammatory compound (NSAID), a retinoid compound, a farnesyl-protein transferase inhibitor, a matrix metalloprotease inhibitor, an αvβ3 integrin inhibitor, an anthracycline compound, an antibody against HER2, and EGFR antagonist or inhibitor, a protein kinase inhibitor, linomide, angiostatin, dehydroepiandrosterone (DHEA), a telomerase inhibitor, a cyclooxygenase inhibitor, razoxin, platelet factor 4 (endostatin), an anti-esrtogen, a VEGF inhibitor and thalidomide, or a mixture thereof, as a combined preparation for simultaneous, separate or sequential use in chemopreventing or controlling the growth of estrogen dependent cancer.

The combination preparation according to the invention can also include combination packs or compositions in which the constituents are placed side by side and can be administered simultaneously, separately of sequentially to one and the same human being. Accordingly, exemestane and the other chemopreventive agent according to the invention may be present within a single or distinct container.

Examples of estrogen hormone dependent cancers are breast, cervical, ovarian and endometrial tumors.

Product exemestane is compound 6-methylenandrost-1,4-diene-3,17-dione, which is known for instance from U.S. Pat. No. 4,808,616.

The term “chemoprevention” is meant to comprise both primary prevention of cancer in people who have not yet developed cancer and secondary prevention of cancer, i.e. the prevention of second primary tumors in patients cured of an initial cancer or the prevention of cancer in people who have had premalignant lesions.

Since cancer usually has a slow, multistep progression , as used herein, “controlling the growth” of estrogen dependent cancer refers to slowing, interrupting or arresting the process at an early precancerous stage in a mammal, including humans, at increased risk of the disease.

The inventors of the present invention have found that combined chemoprevention of estrogen dependent cancer, comprising a therapeutically effective amount of exemestane and a therapeutically effective amount of another chemopreventive agent, as defined above, can produce a therapeutic effect which is greater than that obtainable by single administration of a therapeutically effective amount of either sole exemestane or a sole chemopreventive agent, namely such combined therapy provides a synergistic or superadditive therapeutic effect.

Similarly they have found that a combination chemoprevention therapy of estrogen dependent cancer comprising a therapeutically sub-effective amount of exemestane and a therapeutically sub-effective amount of another chemopreventive agent, as defined above, can produce substantially the same chemoprevention therapeutic effect, which is obtainable by single administration of either exemestane or another chemopreventive agent.

The most important, they have found that such newly obtained therapeutic effect is not paralleled by the toxic effects, otherwise caused by single administration of either therapeutically effective amounts of exemestane or another chemopreventive agent. As stated above, chemopreventive agents are intended for chronic (or long lasting) use in healthy or relative healthy subjects, therefore toxicity, even if mild and reversible, is problematic.

As stated above, the chemoprevention treatment defined herein may be applied as a sole exemestane therapy or may involve, in addition to exemestane one or more chemopreventive agents as defined above. Such conjunct treatment may be achieved by way of the simultaneous, sequential or separate administration of the individual components of the treatment.

A chemopreventive agent mixture, according to the invention, which can be administered in combination with exemestane can comprise: one or more, preferably 1 to 4, in particular 1 or 2, chemopreventive agents, as defined above.

A taxane compound, according to this invention, is e.g. paclitaxel (including liposomal formulations) and docetaxel.

A protein kinase inhibitor, according to the invention, is for instance a tyrosine kinase inhibitor, in particular compound SU6668, i.e. 3-[4-(2-carboxyethyl-3,5-dimethylpyrrol-2-yl)methylidenyl]-2-indolinone, and compound SU5416, i.e. 3-[(2,4-dimethylpyrrol-5-yl)methylidenyl]-2-indolinone, which are known from WO 96/40116 and WO 99/61422. A farnesyl-protein transferase inhibitor, can be for instance one of the inhibitors disclosed in WO 00/25789, in particular (−)-6-[amino(4-chlorophenyl)(1-methyl-1H-imidazol-5-yl)methyl]-4-(3-chlorophenyl)-1-methyl-2(1H)-quinolinone (Compound J-A; designated “comp. 74” in WO 97/21701); (+)-6-[amino(4-chlorophenyl)(1-methyl-1H-imidazol-5-yl)methyl]-4-(3-chlorophenyl)-1-methyl-2(1H)-quinolinone (Compound J-B; designated “comp. 75” in WO 97/21701); and the compound designated as compound “39.0”, which is specifically described in Example 10 of WO 97/23748.

Examples of cyclooxygenase inhibitors are COX-2 inhibitors, in particular celecoxib, rofecoxib, parecoxib and valdecoxib

Examples of retinoid compounds according to the invention include known Accutane; Adapalene; Allergan AGN-193174; Allergan AGN-193676; Allergan AGN-193836; Allergan AGN-193109; Aronex AR-623; BMS-181162; Galderma CD-437; Eisai ER-34617; Etrinate; Fenretinide; Ligand LGD-1550; lexacalcitol; Maxia Pharmaceuticals MX-781; mofarotene; Molecular Design MDI-101; Molecular. Design MDI-301; Molecular Design MDI-403; Motretinide; Eisai 4-(2-[5-(4-methyl-7-ethylbenzofiran-2-yl)pyrrolyl])benzoic acid; Johnson & Johnson N-[4[2-thyl-1-(1H-imidazol-1-yl)butyl]phenyl]-2-benzothiazolamine; Soriatane; Roche SR-11262; Tocoretinate; Advanced Polymer Systems trans-retinoic acid; UAB Research Foundation UAB-8; Tazorac; TopiCare; Taiho TAC-101; and Vesanoid.

Examples of matrix metallo-protease inhibitors according to the invention include known:

1-cyclopropyl-N-hydroxy-4-[[4-[4-(trifluoromethoxy)phenoxy]pheny]sulfonyl]-4-piperidinecarboxamide monohydrochloride;

N-hydroxy-1-(phenylmethyl)-4-[[4-[4-(trifluoromethoxy)phenoxy]-1-piperidinyl]sulfonyl]-4-piperidinecarboxamide monohydrochloride;

N-hydroxy-1-(pyridinylmethyl)-4-[[4-[4-(trifluoromethyl)phenoxy]phenyl]sulfonyl]-4-piperidinecarboxamide dihydrochloride;

N-hydroxy-2,3-dimethoxy-6-[[4-[4-(trifluoromethyl)phenoxy]-1-piperidinyl]sulfonyl]-benzamide;

N-hydroxy-1-(4-pyridinylmethyl)-4-[[4-[4-(trifluoromethyl)phenoxy]phenyl]sulfonyl]-4-piperidinecarboxamide dihydrochloride;

N-hydroxy-1-(3-pyridinylnethyl)-4-[[4-[4-(trifluoromethyl)phenoxy]phenyl]sulfonyl]-4-piperidinecarboxamide dihydrochloride;

N-hydroxy-1-(2-pyridinylmethyl)-4-[[4-[4-(trifluoromethyl)phenoxy]phenyl]sulfonyl]-4-piperidinecarboxamide monohydrochloride;

British Biotech BB-2516 (marimastat), N4-[2,2-dimethyl-1-[(methylamino)carbonyl]-propyl]-N,2-dihydroxy-3-(2-methylpropyl)-, [2S-[N4(R*), 2R*, 3S*]]-);

BMS 275291 disclosed in WO 97/19075;

Bayer Ag Bay-12-9566 (tanomastat), 4-[(4′-chloro[1,1-diphenyl]-4-yl)oxy]-2-[(phenylthio)methyl]butanoic acid;

Agouron Pharmaceuticals AG-3340, N-hydroxy-2,2′-dimethyl-4-[[4-(4-pyridinyloxy)phenyl]sulfonyl]-3-thiomorpholinecarboxamide;

CollaGenex Pharmaceuticals CMT-3 (metastat), 6-demethyl-6-deoxy-4-dedimethylaminotetracycline, batimastat (BB-94); and

Chiroscience D-2163, 2-[1 S-([(2R, S)-acetylnercapto-5-phthalimido]pentanoyl-L -leucyl)amino-3-methylbutyl]imidazole.

Examples of αvβ3 integrin inhibitors are known:

Vitaxin antibody (Ixsys); Merck KgaA EMD-121974, cyclo[RGDF-N(Me)V-];

(10S)-10,11-dihydro-3-[3-(2-pyridinylamino)propoxy]-5H-dibenzo[a,d]cycloheptene-10-acetic acid;

(2S)-7-[[(1H-benzimidazol-2-ylmethyl)methylamino]carbonyl]-2,3,4,5-tetrahydro-4-methyl-3-oxo-1H-1,4-benzodiazepine-2-acetic acid;

(2S)-2,3,4,5-tetrahydro-4-methyl-7-[[[(5-methyl-1H-imidazo[4,5-b]pyridin-2-yl]methyl]amino]carbonyl]-3-oxo-1H-1,4-benzodiazepine-2-acetic acid;

(bR)-b-[[[(3R)-2-oxo-3-[2-(5,6,7,8-tetrahydro-[1,8]-naphthyridin-2-yl)ethyl] 1-1-pyrrolidinyl]acetyl]amino]-d-(1H-indol-3-yl)pentanoic acid; and

(3R)-N-[3-hydroxy-5-[(1,4,5,6-tetrahydro-5-hydroxy-2-pyrimidinyl)amino]benzoyl]-glycyl-3-(3-bromo-5-chloro-2-hydroxyphenyl)-b-alanine (compound SD 7784).

An antracycline compound, according to the invention is e.g. doxorubicin (including liposomal formulations), epirubicin (including liposomal formulations), idarubicin, nemorubicin, daunomycin, mitomycin-C, dactinomycin and mithramycin.

An EGFR inhibitor is for instance compound CP-358,774 and ZD 1839, which are known e.g. from Proceedings of ASCO volume 18, 1999 page 388a, and ZM.254530, which is known from WO 95/03283.

An EGFR antagonist is for instance an antibody, in particular chimerized antibody C225 and human antibodies E1.1, E2.4, E2.5, E6.2, E6.4, E2.11, E6.3 and E7.6.3, in particular E7.6.3. Preferred antibodies against EGFR are chimerized antibody C225 and human antibody E7.6.3. Chimerized antibody C225 is disclosed by WO96/49210. Human antibodies E1.1, E2.4, E2.5, E6.2, E6.4, E2.11, E6.3 and E7.6.3 are disclosed by WO 98/50433.

An antibody against HER2 can be either an “intact” antibody or a fragment thereof, e.g. Fab, Fab′, F(ab′)2 or Fv fragments. A preferred example of an antibody against HER2 is trastuzumab, which is described e.g. in Cancer Res., 1998, 58:2825-2831.

A non-steroidal anti-inflammatory compound (NSAID), according to the invention, is e.g. a compound selected from acetyl salicylic acid, indometacin, sulindac, phenylbutazone, diclofenac, fentiazac, ketorolac, piroxicam, tenoxicam, mecoxicam, cinnoxicam, ibufenac, ibuprofen, naproxen, ketoprofen, nabumetone, niflumic acid and nimesulide, or a pharmaceutically acceptable salt thereof. Preferred NSAIDs are diclofenac, piroxicam, tenoxicam, mecoxicam, ibufenac, ibuprofen, naproxen and ketoprofen, or a pharmaceutically acceptable salt thereof

An anti-estrogen, e.g. a selective estrogen receptor modulator (SERM), is preferably selected from tamoxifen, raloxifene, toremifene, arzoxifene, idoxifene, EM 800, fulvestrant and droloxifene.

Vascular endothelial growth factor (VEGF) inhibitors and telomerase inhibitors are well known in the art. For instance, compounds SU 5416 and SU 6668, cited herein, are also VEGF inhibitors.

Moreover known VEGF inhibitors or antagosts are i.e. agents which suppress angiogenesis by reducing binding of VEGF to cellular receptors, including but not limited to, for example blocking monoclonal antibodies against the growth factor (e.g. rhuMAbVEGF, Ryan et al., Toxicol Pathol 1999, 27:78-86), against the receptor (e.g. DC101 and derivatives, Witte et al., Cancer Metastasis Rev 1998, 17:155-61), soluble forms of VEGF receptors (e.g. soluble Flt, Aiello et al., Proc Natl Acad Sci USA 1995, 92:10457-61), or compounds which directly antagonise interactions between VEGF and cell surface receptors (e.g. Fairbrother et al., Biochemistry 1998, 37:17754-64). Linomide, razoxyn and thalidomide are known antiangiogenetic agents.

In order to identify women at high risk for the development of hormone dependent cancer (and therefore in need of chemo-prevention) tumor markers, tumor biomarkers and surrogate endpoint tissue biomarkers (SEBs) commonly used in clinical hormone dependent cancer diagnosis can be employed.

The term “tumor marker” or “tumor biomarker” or “SEBs” in its broad meaning encompasses a wide variety of molecules with divergent characteristics that appear in body fluids or tissue in association with a clinical tumor and also includes tumor-associated chromosomal changes. Tumor markers fall primarily into three categories: molecular or cellular markers, chromosomal markers, and serological or serum markers. In particular, as to serum markers, they can often be measured many months before clinical tumor detection and are useful as an early diagnostic test, in patient monitoring, and therapy evaluation.

For instance in primary chemo-prevention of breast cancer, as SEBs the following can be used: Generic Markers: routine histopathology, morphology, proliferation, neovascularization; and Specific markers: estrogen receptors (ER), Progesteron receptors, ErbB2, EGFR, VGFR, BCRA-1, BCRA-2, PS2 and IGFR1R

As to SEBs in secondary chemoprevention of breast cancer, for instance the following can be used: epithelial hyperplasia without atypia or with atypia, as well as abnormalities of several cellular biomarkers (DNA ploidy, p53, EGFR, ER, PgR, and her2/neu). Increasing cytologic abnormality is in general associated with increasing frequency of biomarkers abnormalities, and evidence of a typical hyperplasia plus multiple biomarkers abnormalities is the most common presentation for women who subsequently develops cancer. Also increased mammographic density has been associated with an increased risk of breast cancer and therefore mammographic density can represent a suitable SEB. In addition, breast magnetic resonance imaging (MRI) can be an important SEB.

Mammals, including humans, in particular women, who have rising tumor markers but no clinical evidence of the disease are therefore at risk of the disease. Accordingly in such mammals the multi-step progression that leads to cancer can be slowed, interrupted or arrested at an early pre-cancerous stage by the chemo-prevention therapy method provided by the present invention.

Pharmacology

The therapeutic effect of exemestane either alone or in combination with another chemopreventive agent, according to the invention, in the hormone-dependent cancer in mammals is proven, for instance by the fact that exemestane has been found to be active in the prevention of the dimethylbenzanthracene (DMBA)-induced mammary tumor model in rats. Exemestane treatment (4, 20 or 100 mg/kg/wk, IM) started 1 week after DMBA exposure (20 mg/rat, PO) and continued for 19 weeks. At the end of the 19-week treatment period, exemestane significantly decreased tumor incidence from 85% in vehicle treated rats to 13.6% in the 100 mg/kg treated group. Moreover, exemestane at 100 mg/kg reduced significantly the tumor multiplicity, being 2.55 the number of tumors/rat in the control groups versus 0.27 in the treated group. No signs of toxicity were observed.

Method and Administration

In effecting treatment of a patient in a therapy/prophylactic method according to the invention, exemestane and the other chemopreventive agent can be administered in any form or mode which makes the compounds bioavailable in effective amounts, including oral and parenteral routes.

By the term “administered” or “administering” as used herein is meant any acceptable manner of administering a drug to a patient which is medically acceptable including parenteral and oral administration.

By “parenteral” is meant intravenous, subcutaneous, intradermal or intramuscular administration.

Oral administration includes administering the constituents of the combined preparation in a suitable oral form such as, e.g., tablets, capsules, suspensions, solutions, emulsions, powders, syrups and the like.

The actual preferred method and order of administration of the combined preparations of the invention may vary according to, inter alia, the particular pharmaceutical formulation of exemestane being utilized, the particular pharmaceutical formulation of the other chemopreventive agent being utilized, the particular cancer to be prevented and the particular patient being treated.

The dosage ranges for the administration of the combined preparation may vary with the age, condition and extent of the disease in the patient and can be determined by one of skill in the art.

The dosage regimen must therefore be tailored to the particular of the patient's conditions, response and associate treatments in a manner which is conventional for any therapy, and may need to be adjusted in response to changes in conditions and/or in light of other clinical conditions.

In the combined method of treatment according to the subject invention, exemestane may be administered simultaneously with the other chemopreventive agent or the compounds may be administered sequentially, in either order.

Dosage

According to the chemoprevention method of estrogen dependent cancers in mammals, provided the present invention, exemestane for instance can be administered orally in a dosage range varying from about 5 mg daily to about 600 mg daily, in particular from about 10 to about 50, more preferably about 25 mg daily, or intramuscularly in a dosage ranging from about 50 to about 500 mg per injection. As a preferred embodiment of the invention, exemestane is orally administered in the form of a complex with cyclodextrins, in particular exemestane/β-cyclodextrin complex, at a daily dosage ranging from about 10 to about 20 mg, preferably about 15 or 20 mg.

The effective therapeutic amounts of the other chemopreventive agents to be used in combination with exemestane, according to the invention, are in general those commonly used in therapy for such compounds. More specifically, a therapeutically effective amount of another chemopreventive agent means an amount of a compound, which when administered in combination with exemestane, is effective to prevent estrogen dependent cancers.

Determination of a therapeutically effective amount is well within the capability of those skilled in the art. For instance an effective amount of compound SU 5416 or SU 6668 is an amount in accordance with the teaching of WO 99/61422.

An effective amount of compound SD 7784 is from about 10 to about 300 mg/kg, preferably per os, in particular from about 20 to about 200 mg/kg.

An effective chemopreventive amount of doxorubicin may vary from about 20 mg/M² to about 100 mg/m².

An effective chemopreventive amount of epirubicin may vary from about 20 mg/m² to about 200 mg/m².

An effective chemopreventive amount of idarubicin may vary from about 1 mg/m² to about 50 mg/m².

An effective chemopreventive amount of paclitaxel may vary from about 100 mg/m² to about 300 mg/m².

An effective chemopreventive amount of docetaxel may vary from about 50 mg/m² to about 100 mg/m².

A chemopreventive amount, for example for recombinant humanized monoclonal antibody anti-HER2 trastuzumab, is from about 1 to about 1000 mg/m² of body surface area. More preferably, the course therapy employed is from about 50 to about 500 mg/m² of body surface area.

In the method of the subject invention, for example for the administration of the recombinant humanized monoclonal antibody anti-EGFR C225 (cetuximab), the course of therapy generally employed is from about 150 to about 500 mg/m² of body surface area. Preferably, the course therapy employed consists of a loading dose of about 400 mg/m², followed by weekly maintenance dosage of about 180-250 mg/m². According to a preferred embodiment of the invention patients are given an injection of cetuximab as a weekly, dose escalating 4-week protocol, with doses up to 200 mg/m². If the disease is stabilized, then a further 8-week course can begin.

In the method of the subject invention, for the administration e.g. of the recombinant humanized monoclonal antibody E7.6.3 the course of therapy generally employed is from about 1 to about 1000 mg/m² of body surface area. More preferably, the course therapy employed is from about 60 to about 600 mg/m² of body surface area.

In the method of the subject invention, for the administration e.g. of compound CP-358774 the course of therapy generally employed is from about 25 to about 150 mg/day p.os., so that to reach a plasma concentration from about 300 to about 700 ng/ml, preferably 500 ng/ml.

In the method of the subject invention, for the administration e.g. of compound ZD 1839 the course of therapy generally employed is from about 50 to about 300 mg/day p.os.

An anti-etrogen can be administered in a dosage according to the common practice, e.g. in a dosage of about 0.1 to about 30 mg/Kg body weight per day.

An effective amount of a COX-2 inhibitor may be in the range of about 0.1 to about 2000 mg, preferably in the range of about 0.5 to about 500 and most preferably between about 1 and about 200 mg. In particular as to celecoxib, rofecoxib, parecoxib and valdecoxib, a daily dosage of about 0.01 to about 100 mg/Kg boyd weight, preferably between about 0.1 and about 50 mg/Kg body weight may be appropriate. The daily dosage can be administered in one to four doses per day.

More particularly, as to celecoxib a dosage from about 50 to about 500 mg, in particular about 200 mg, once or twice a day may be appropriate.

As to rofecoxib the dosage normally ranges from about 12.5 to about 50 mg/day. The route of administration is preferably systemic e.g. oral or parenteral, in particular intravenous or intramuscularly.

From the pharmacological point of view, the valuable biological properties of exemestane may be found in its peculiar mechanism of aromatase inactivation.

The aromatase enzyme (450ₐᵣₒₘ) is a specific form of cytochrome P450 hemoprotein composed of a P450 (heme) moiety and a peptidic moiety. The enzyme catalyzes a multistep reaction leading to aromatization of the A ring of the androgen substrate (mainly androstenedione) to estrone, requiring the presence of the cofactor NADPH. After this enzymatic reaction, the enzyme molecule is once more available to perform a new aromatization.

The exemestane's mechanism of aromatase inhibition has been extensively studied and the compound has been found to cause enzyme inactivation. In fact exemestane, structurally related to the natural substrate androstenedione, is initially recognized by the aromatase enzyme as a false substrate, therefore competes with androstenedione at the active site of the enzyme. The compound is then transformed (through and NADPH-dependent mechanism) to an intermediate which binds irreversibly to the enzyme causing its inactivation (also known as suicide inhibition). Therefore the enzyme is definitely inactivated and de novo enzyme synthesis is required for oestrogen production.

Therefore, the compositions and combined therapy method of the invention, thanks to the biological activity of exemestane as aromatase inactivator and the different biological activity of the additional chemopreventive agent, provide a two-way attack of cancer. Suitable modifications and adaptations of a variety of conditions and parameters normally encountered in clinical therapy which are obvious to those skilled in the art are within the scope of this invention.

A pharmaceutically composition containing exemestane and/or another chemopreventive agent according to the invention can be prepared according to well known techniques to those skilled in the art. For instance a pharmaceutical composition containing exemestane can be prepared according to U.S. Pat. No. 4,808,616.

As to exemestane/cyclodextrin complex, it has to be noticed that cyclodextrins are crystalline, water soluble, cyclic, non-reducing oligosaccharides built up six, seven, or eight glucopyranose units that have a cylindrical cavity shaped structure capable of including various guest molecules. Due to their peculiar structure, one of the most interesting features of cyclodextrins is their ability to form inclusion compounds or complexes. At the pharmaceutical level, the applications of these inclusions are essentially for improving the stability and above all the solubility, dissolution characteristics and potentially the bioavailability of the included molecule, thus allowing the deliverability of difficult to formulate actives or a significant improvement of their biopharmaceutical properties.

Cyclodextrin/drug complexes offer two important product advantages for oral preparations: improved bioavailability and reduced irritation.

Improved bioavailability is observed for certain drugs which are metabolized in the gastro intestinal tract, or are not fully absorbed or are absorbed in a variable manner due to incomplete dissolution of the drug in the gastrointestinal tract.

CDs offer the potential for improving the reliability of oral dosing by permitting the use of true solutions of the drug rather than suspensions during manufacture of the tablets or as the final formulation available to the patient.

All the above mentioned factors and prospected advantages are particularly true as far as steroidal drug formulations for the oral route are concerned.

If fact it is well known that steroidal actives (as exemestane is) suffers for both pre- systemic extraction (through degradation in the gastro-intestinal environment and first pass hepatic effects) and poor biopharmaceutical properties (being their acqueous solubility in most of the cases negligible).

Thus, the complexation of the active with an agent able to improve the physico-chemical properties of the active and to protect it from the external environment (such as a cyclodextrin) potentially allow to administer unit dosage formulas that contain a lower amount of: active drug substance, without any detrimental effect on its availability and clinical efficacy.

As far as exemestane is concerned, it was experimentally verified that a 1:2 molar ratio inclusion complex between the active and beta-cyclodextrin improve 7 times the solubility of the active, 9 times its intrinsic dissolution rate and significantly its chemical stability, reinforcing the possibility to have formulations not only more stable, as far as the shelf-life is concerned, but also more promptly and effectively bioavailable.

The coupling of all these factors allow to obtain the same clinical efficacy by administering lower quantities of the active.

As example, if hypothetically the bioavailability of a exemestane/beta-cyclodextrin formulation is 30% higher in comparison to the one of a conventional formula (i.e. the sugar coated formula currently marketed as Aromasin™), the daily administration dose necessary to gain the same clinical efficacy can be reduced from 25 to 10-20 mg.

This is of particular interest for therapeutic applications such as chemoprevention, where the drug has to administered chronically for extremely long durations. Formulation example:

Exemestane 20 mg Tablet

| | | |
|---|---|---|
| Composition: | exemestane | 20.00 mg |
| | Beta-cyclodextrin | 178.00 mg |
| | Avicel PH101 | 75.00 mg |
| | Explotab | 24.00 gm |
| | Magnesium stearate | 3.00 mg |

According to methods well known in the art an exemestane/cyclodestrin kneaded system can be prepared.

The inventors of this invention have found that the combined treatment of exemestane and another therapeutic agent, as herein defined, besides being active in preventing, is also active in treating estrogen dependent cancers, in particular the cancers mentioned above. Moreover, by such combined treatment a synergistic or superadditive antitumor effect can be provided.

Accordingly, the present invention also provides the use of exemestane in the manufacture of a medicament for treating estrogen dependent cancer, in a patient undergoing a simultaneous, separate of sequential therapy, with another therapeutic agent selected from a non-steroidal anti-inflammatory compound (NSAID), a retinoid compound, a farnesyl-protein transferase inhibitor, a matrix metalloprotease inhibitor, an αvβ3 integrin inhibitor, a protein kinase inhibitor, linomide, angiostatin, dehydroepiandrosterone (DHEA), a telomerase inhibitor, platelet factor 4 (endostatin), toremifene, droloxifene, a cyclooxygenase inhibitor, SU 5416, SU 6668, razoxyn, arzoxifene, idoxifene, fluvestrant, EM 800 and thalidomide, or a mixture thereof.

The invention in addition provides a combined method for treating estrogen dependent cancer in a mammal in need of such treatment, including humans, comprising administering simultaneous, separately or sequentially to said mammal, exemestane, and another therapeutic agent selected from a non-steroidal anti-inflammatory compound (NSAID), a retinoid compound, a farnesyl-protein transferase inhibitor, a matrix metalloprotease inhibitor, an αvβ3 integrin inhibitor, a protein kinase inhibitor, linomide, a cyclooxygenase inhibitor, SU 5416, SU 6668, razoxyn, angiostatin, dehydroepiandrosterone (DHEA), a telomerase inhibitor, platelet factor 4 (endostatin), toremifene, droloxifene, arzoxifene, idoxifene, fluvestrant, EM 800 and thalidomide, or a mixture thereof; in amounts and close in time sufficient to produce a therapeutically useful effect.

The invention also provides a product containing exemestane and another therapeutic agent selected from a non-steroidal anti-inflammatory compound (NSAID), a retinoid compound, a farnesyl-protein transferase inhibitor, a matrix metalloprotease inhibitor, an αvβ3 integrin inhibitor, a protein kinase inhibitor, linomide, angiostatin, a cyclooxygenase inhibitor, SU 5416, SU 6668, razoxyn, dehydroepiandrosterone (DHEA), a telomerase inhibitor, platelet factor 4 (endostatin), toremifene, droloxifene, arzoxifene, idoxifene, fluvestrant, EM 800 and thalidomide, or a mixture thereof, as a combined preparation for simultaneous, separate or sequential use in treating estrogen dependent cancer.

The combination preparation according to the invention can also include products, namely combination packs or compositions, in which the constituents are placed side by side and can be administered simultaneously, separately of sequentially to one and the same human being. Accordingly, exemestane and the other therapeutic agent according to the invention may be present within a single or distinct container.

By the term “a superadditive or synergistic antitumor effect” as used herein is meant the inhibition of the growth tumor, preferably the complete regression of the tumor, by administering a combination of exemestane and another therapeutic agent, to a human being, particularly a human female.

Said preparation having therefore a potentiated antitumor (superadditive) activity with respect to products containing either exemestane or the other therapeutic agent, which is greater than the sum of the actions of individual components.

According to a preferred aspect of the present invention the superadditive antitumor effect results in an anti-cancer therapy having increased effectiveness in controlling, i.e. slowing, interrupting, arresting, stopping or reversing, the neoplasm formation.

As used herein, “controlling the growth” of the neoplasm refers to slowing, interrupting, arresting or stopping its growth and it does not necessarily indicate a total elimination of the neoplasm.

Therefore, the term “treating” simply means that the life expectancy of an individual affected with a cancer will be increased, that one or more of the symptoms of the disease will be reduced and/or that quality of life will be enhanced.

1. Use of exemestane in the manufacture of a medicament for chemoprevention or controlling the growth of estrogen dependent cancer.

2. Use, according to claim 1, wherein the medicament is for primary prevention of estrogen dependent cancer.

3. Use, according to claim 1, wherein the medicament is for secondary prevention of estrogen dependent cancer.

4. Use, according to claim 1, wherein the estrogen dependent cancer is breast, cervical, ovarian or endometrial tumor.

5. Use, according to claim 1, wherein the medicament is for oral administration and the exemestane content is from about 10 to about 50 mg; or the medicament is for intramuscular administration and the exemestane content is from about 50 to about 600 mg.

6. Use of exemestane in the manufacture of a medicament for chemoprevention or controlling the growth of estrogen dependent cancer in a patient undergoing a simultanous, separate or sequential treatment, with another chemopreventive agent selected from a taxane compound, a non-steroidal anti-inflammatory compound (NSAID), a retinoid compound, a farnesyl-protein transferase inhibitor, a matrix metalloprotease inhibitor, an αvβ3 integrin inhibitor, an anthracycline compound, an antibody against HER2, and EGFR antagonist or inhibitor, a protein kinase inhibitor, linomide, angiostatin, dehydroepiandrosterone (DHEA), a telomerase inhibitor, a cyclooxygenase inhibitor, razoxyn, platelet factor 4 (endostatin), an anti-estrogen, a VEGF inhibitor and thalidomide, or a mixture thereof.

7. Use, according to claim 6, wherein a superadditive therapeutic effect is provided.

8. Use, according to claim 6, wherein the medicament is for primary prevention of estrogen dependent cancer.

9. Use, according to claim 6, wherein the medicament is for secondary prevention of estrogen dependent cancer.

10. Use, according to claim 6, wherein the estrogen dependent cancer is breast, cervical, ovarian or endometrial tumor.

11. Use, according to claim 6, wherein a mixture of chemopreventive agents, to be administered in combination with exemestane, comprises 1 to 4 chemopreventive agents as defined in claim 6.

12. Use, according to claim 6, wherein the taxane compound is selected from: paclitaxel (including liposomal formulations) and docetaxel.

13. Use, according to claim 6, wherein the protein kinase inhibitor is selected from: 3-[4-(2-carboxyethyl-3,5-dimethylpyrrol-2-yl)methylidenyl]-2-indolinone, and 3-[(2,4-dimethylpyrrol-5-yl)methylidenyl]-2-indolinone.

14. Use, according to claim 6, wherein the farnesyl-protein transferase inhibitor is selected from:
(−)-6-[amino(4-chlorophenyl)(1-methyl-1H-imidazol-5-yl)methyl]-4-(3-chlorophenyl)-1-methyl-2(1H)-quinolinone,
(+)-6-[amino(4-chlorophenyl)(1-methyl-1H-imidazol-5-yl)methyl]-4-(3-chlorophenyl)-1-methyl-2(1H)-quinolinone, and
Compound “39.0”.

15. Use, according to claim 6, wherein the retinoid compound is selected from: Accutane; Adapalene; Allergan AGN-193174; Allergan AGN-193676; Allergan AGN-193836; Allergan AGN-193109; Aronex AR-623; BMS-181162; Galderma CD-437; Eisai ER-34617; Etrinate; Fenretinide; Ligand LGD-1550; lexacalcitol; Maxia Pharmaceuticals MX-781; mofarotene; Molecular Design MDI-101; Molecular Design MDI-301; Molecular Design MDI-403; Motretinide; Eisai 4-(2-[5-(4-methyl-7-ethylbenzofuran-2-yl)pyrrolyl])benzoic acid; Johnson & Johnson N-[4-[2-thyl-1-(1H-imidazol-1-yl)butyl]phenyl]-2-benzothiazolamine; Soriatane; Roche SR-11262; Tocoretinate; Advanced Polymer Systems trans-retinoic acid; UAB Research Foundation UAB-8; Tazorac; TopiCare; Taiho TAC-101; and Vesanoid.

16. Use, according to claim 6, wherein the metallo-protease inhibitor is selected from:1-cyclopropyl-N-hydroxy-4-[[4-[4-(trifluoromethoxy)phenoxy]phenyl]sulfonyl]-4-piperidinecarboxamide monohydrochloride;
N-hydroxy-1-(phenylmethyl)-4-[[4-[4-(trifluoromethoxy)phenoxy]-1-piperidinyl]sulfonyl]-4-piperidinecarboxamide monohydrochloride;
N-hydroxy-1-(pyridinylmethyl)-4-[[4-[4-(trifluoromethyl)phenoxy]phenyl]sulfonyl]-4-piperidinecarboxamide dihydrochloride;
N-hydroxy-2,3-dimethoxy-6-[[4-[4-(trifluoromethyl)phenoxy]-1-piperidinyl]sulfonyl]-benzamide;
N-hydroxy-1-(4-pyridinylmethyl)-4-[[4-[4-(trifluoromethyl)phenoxy]phenyl] sulfonyl]-4-piperidinecarboxamide dihydrochloride; N-hydroxy-1-(3-pyridinylmethyl)-4-[[4-[4-(trifluoromethyl)phenoxy]phenyl]sulfonyl]-4-piperidinecarboxamide dihydrochloride;
N-hydroxy-1-(2-pyridinylmethyl)-4-[[4-[4-(trifluoromethyl)phenoxy]phenyl]sulfonyl]-4-piperidinecarboxamide monohydrochloride;
British Biotech BB-2516 (Marimastat), N4-[2,2-dimethyl-1-[(methylamino)carbonyl]-propyl]-N1,2-dihydroxy-3-(2-methylpropyl)-, [2S-[N4(R*), 2R*, 3S*]]-);
BMS 275291;
Bayer Ag Bay-12-9566 (tanomastat), 4-[(4′-chloro[l,l-diphenyl]-4-yl)oxy]-2-[(phenylthio)methyl]butanoic acid;
Agouron Pharmaceuticals AG-3340, N-hydroxy-2,2′-dimethyl-4-[[4-(4-pyridinyloxy)phenyl]sulfonyl]-3-thiomorpholinecarboxamide;
CollaGenex Pharmaceuticals CMT-3 (Metastat), 6-demethyl-6-deoxy-4-dedimethylaminotetracycline; batimastat (BB-94); and
Chiroscience D-2163, 2-[1S-([(2R, S)-acetylmercapto-5-phthalimido]pentanoyl-L -leucyl)amino-3-methylbutyl]imidazole.

17. Use, according to claim 6, wherein the αvβ3 integrin inhibitor is selected from:
Vitaxin antibody (Ixsys); Merck KgaA EMD-121974, cyclo[RGDF-N(Me)V-];
(10S)-10,11-dihydro-3-[3-(2-pyridinylamino)propoxy]-5H-dibenzo[a,d]cycloheptene-10-acetic acid;
(2S)-7-[[(1H-benzimidazol-2-ylmethyl)methylamino]carbonyl]-2,3,4,5-tetrahydro-4-methyl-3-oxo-1H-1,4-benzodiazepine-2-acetic acid;
(2S)-2,3,4,5-tetrahydro-4-methyl-7-[[[(5-methyl-1H-imidazo[4,5-b]pyridin-2-yl]methyl]amino]carbonyl]-3-oxo-1H-1,4-benzodiazepine-2-acetic acid;
(bR)-b-[[[(3R)-2-oxo-3-[2-(5,6,7,8-tetrahydro-[1,8]-naphthyridin-2-yl)ethyl]1-1-pyrrolidinyl]acetyl]amino]-d-(1H-indol-3-yl)pentanoic acid; and
(3R)-N-[3-hydroxy-5-[(1,4,5,6-tetrahydro-5-hydroxy-2-pyrimidinyl)amino]benzoyl]-glycyl-3-(3-bromo-5-chloro-2-hydroxyphenyl)-b-alanine (compound SD 7784).

18. Use, according to claim 6, wherein the antracycline compound is selected from: doxorubicin (including liposomal formulations), epirubicin (including liposomal formulations), idarubicin, nemorubicin, daunomycin, mitomicin-C, dactimomycin and mithramycin.

19. Use, according to claim 6, wherein the EGFR inhibitor is selected from compound CP-358,774, ZD 1839, and ZM.254530.

20. Use, according to claim 6, wherein the EGFR antagonist is selected from: chimerized antibody C225 and human antibodies E1.1, E2.4, E2.5, E6.2, E6.4, E2.11, E6.3 and E7.6.3.

21. Use, according to claim 6, wherein the antibody against HER2 is trastuzumab.

22. Use, according to claim 6, wherein the anti-estrogen is selected from tamoxifen, raloxifene, toremifene, arzoxifene, idoxifene, fluvestrant, EM 800 and droloxifene.

23. Use, according to claim 6, wherein the NSAID is selected from acetyl salicylic acid, indometacin, sulindac, phenylbutazone, diclofenac, fentiazac, ketorolac, piroxicam, tenoxicam, mecoxicam, cinnoxicam, ibufenac, ibuprofen, naproxen, ketoprofen, nabumetone, niflumic acid and nimesulide, or a pharmaceutically acceptable salt thereof.

24. Use, according to claim 6, wherein the cyclooxygenase inhibitor is selected from celecoxib, rofecoxib, parecoxib and valdecoxib.

25. Use of exemestane in the manufacture of a medicament in the form of an exemestane/cyclodextrin complex to be administered orally for chemoprevention or controlling the growth of estrogen dependent cancer.

26. Use, according to claim 25, wherein the exemestane amount in the exemestane/cyclodextrin complex is about 15 mg.

27. Use, according to claim 25, wherein the exemestane amount in the exemestane/cyclodextrin complex is about 20 mg.

28. Use of exemestane in the manufacture of a medicament for treating estrogen dependent cancer in a patient undergoing a simultaneous, separate or sequential treatment with another therapeutic agent selected from a non-steroidal anti-inflammatory compound (NSAID), a retinoid compound, a farnesyl-protein transferase inhibitor, a matrix metalloprotease inhibitor, an αvβ3 integrin inhibitor, a protein kinase inhibitor, linomide, angiostatin, dehydroepiandrosterone (DHEA), a telomerase inhibitor, a platelet factor 4 (endostatin), arzoxifene, idoxifene, a cyclooxygenase inhibitor, SU 5416, SU 6668, razoxyn, fluvestrant, EM 800 and thalidomide, or a mixture thereof.

29. Use, according to claim 28, wherein the estrogen dependent cancer is breast, cervical, ovarian or endometrial tumor.

30. Product containing exemestane and another therapeutic agent selected from a non-steroidal anti-inflammatory compound (NSAID), a retinoid compound, a farnesyl-protein transferase inhibitor, a matrix metalloprotease inhibitor, an αvβ3 integrin inhibitor, a protein kinase inhibitor, linomide, angiostatin, dehydroepiandrosterone (DHEA), a telomerase inhibitor, a platelet factor 4 (endostatin), arzoxifene, idoxifene, a cyclooxygenase inhibitor, SU 5416, SU 6668, razoxyn, fluvestrant, EM 800 and thalidomide, or a mixture thereof, as a combined preparation for simultaneous, separate or sequential use in treating estrogen dependent cancer.

31. Product containing exemestane and another chemopreventive agent selected from a taxane compound, a non-steroidal anti-inflammatory compound (NSAID), a retinoid compound, a farnesyl-protein transferase inhibitor, a matrix metalloprotease inhibitor, an αvβ3 integrin inhibitor, an anthracycline compound, an antibody against HER2, and EGFR antagonist or inhibitor, a protein kinase inhibitor, linomide, angiostatin, a cyclooxygenase inhibitor, razoxin, dehydroepiandrosterone (DHEA), a telomerase inhibitor, platelet factor 4 (endostatin), an anti-estrogen, a VEGF inhibitor and thalidomide, or a mixture thereof, as a combined preparation for simultaneous, separate or sequential use in chemopreventing and controlling the growth of estrogen dependent cancer.

32. Method for chemopreventing or controlling the growth of estrogen dependent cancer in a mammal in need of such treatment, including humans, comprising administering to said mammal a therapeutically effective amount of exemestane.

33. Method, according to claim 32, wherein exemestane is administered orally in the form of exemestane/cyclodextrin complex.

34. Method, according to claim 33, wherein exemestane is administered at a daily dosage of about 15 mg.

35. Method, according to claim 33, wherein exemestane is administered at a daily dosage of about 20 mg.

36. The method, according to claim 32, wherein about 5 to 600 mg/day of exemestane is adminsitered orally.

37. The method, according to claim 32, wherein about 10 to 50 mg/day of exemestane is administered orally.

38. The method, according to claim 32, wherein about 25 mg/day of exemestane is administered orally.

39. The method, according to claim 32, wherein about 50 to 500 mg/day of exemestane is administered parenterally.

40. Method for chemopreventing or controlling the growth of estrogen dependent cancer in a mammal in need of such treatment, including humans, comprising administering simultaneous, separately or sequentially to said mammal, exemestane, and another chemopreventive agent selected from a taxane compound, a non-steroidal anti-inflammatory compound (NSAID), a retinoid compound, a farnesyl-protein transferase inhibitor, a matrix metalloprotease inhibitor, an αvβ3 integrin inhibitor, an anthracycline compound, an antibody against HER2, and EGFR antagonist or inhibitor, a protein kinase inhibitor, linomide, angiostatin, dehydroepiandrosterone (DHEA), a telomerase inhibitor, platelet factor 4 (endostatin), an anti-estrogen, a cyclooxygenase inhibitor, razoxyn, a VEGF inhibitor and thalidomide, or a mixture thereof; in amounts and close in time sufficient to produce a therapeutically useful effect.

41. Method, according to claim 40, wherein exemestane and the other chemopreventive agent are administered simultaneously.

42. Method, according to claim 40, wherein exemestane and the other chemopreventive agent are administered sequentially.

43. The method, according to claim 40, wherein about 5 to 600 mg/day of exemestane is administered orally.

44. The method, according to claim 40, wherein about 10 to 50 mg/day of exemestane is administered orally.

45. The method, according to claim 40, wherein about 25 mg/day of exemestane is administered orally.

46. The method, according to claim 40, wherein about 50 to 500 mg/day of exemestane is administered parenterally.

47. Method, according to claim 40, wherein the anti-estrogen is selected from tamoxifen, raloxifene, toremifene, arzoxifene, idoxifene, faslodex, EM 800 and droloxifene.

48. Method according to claim 40, wherein the COX-2 inhibitor is selected from celecoxib, rofecoxib, parecoxib and valdecoxib.

49. Method according to claim 40, wherein the protein kinase inhibitor is selected from 3-[4-(2-carboxyethyl-3,5-dimethylpyrrol-2-yl)methylidenyl]-2-indolinone, and 3-[(2,4-dimethylpyrrol-5-yl)methylidenyl]-2-indolinone.

50. Method for treating estrogen dependent cancer in a mammal in need of such treatment, including humans, comprising administering simultaneous, separately or sequentially to said mammal, exemestane, and another therapeutic agent selected from a non-steroidal anti-inflammatory compound (NSAID), a retinoid compound, a farnesyl-protein transferase inhibitor, a matrix metalloprotease inhibitor, an αvβ3 integrin inhibitor, a protein kinase inhibitor, linomide, angiostatin, a cyclooxygenase inhibitor, SU 5416, SU 6668, razoxyn, dehydroepiandrosterone (DHEA, a telomerase inhibitor, a platelet factor 4 (endostatin), arzoxifene, idoxifene, fluvestrant, EM 800 and thalidomide, or a mixture thereof; in amounts and close in time sufficient to produce a therapeutically useful effect.

51. A method according to claim 50 wherein the cyclooxygenase inhibitor is selected from celecoxib, parecoxib, rofecoxib and valdecoxib.

52. Method, according to claim 50, wherein exemestane and the other chemopreventive agent are administered simultaneously.

53. Method, according to claim 50, wherein exemestane and the other chemopreventive agent are administered sequentially.
